(19) United States
(12) Patent Application Publication
(10) Pub. No.: US 2015/0259628 A1
(43) Pub. Date: Sep. 17, 2015

(54) AROMATIC SUBSTANCE MIXTURES CONTAINING HEXYL SALICYLATE

(71) Applicant: Henkel AG & Co. KGaA, Duesseldorf (DE)

(72) Inventors: Georg Meine, Mettmann (DE); Ralf Bunn, Duesseldorf (DE); Hubert Smyrek, Krefeld (DE); Andrea Eutebach, Wuppertal (DE); Frank Sonnenschein, Haan (DE)

(21) Appl. No.: 14/722,631

(22) Filed: May 27, 2015

Related U.S. Application Data
(63) Continuation of application No. PCT/EP2013/073369, filed on Nov. 8, 2013.

(30) Foreign Application Priority Data
Nov. 27, 2012 (DE) 10 2012 221 619.7

(51) Int. Cl. C11D 3/50; A61Q 15/00; A61Q 19/00; C11B 9/00; A61Q 9/02; A61Q 19/10; A61Q 5/02; C11D 3/20; A61K 8/37

(52) U.S. Cl. CPC C11D 3/50; C11D 3/2093; A61Q 15/00; A61Q 19/00; A61K 8/37; A61Q 9/02; A61Q 19/10; A61Q 5/02; C11B 9/0061

(57) ABSTRACT

The invention relates to odorant mixtures containing hexyl salicylate and at least one other salicylate, to agents which contain corresponding odorant mixtures, to the use of odorant mixtures containing salicylates and of agents for reducing unpleasant odors, and to a method for manufacturing a detergent or cleaning agent, fabric conditioner, hygienic rinse, or dryer sheet for reducing unpleasant odors.

### FIELD OF THE INVENTION

The present invention generally relates to odorant mixtures containing salicylate, to agents which contain corresponding odorant mixtures, to the use of odorant mixtures containing salicylate and of agents for reducing unpleasant odors, and to a method for manufacturing a detergent or cleaning agent, fabric softener, hygienic rinse, or dryer sheet which reduces unpleasant odors.

### BACKGROUND OF THE INVENTION

Even after a thorough washing or cleaning, germs remain on a solid or textile surface or also on the human skin in a small number which is not critical to health or perceivable via the sense of smell. However, if these germs have the opportunity to multiply with sufficient heat and moisture, within a very short time the number of germs on the surface increases to the extent that the germs or their reaction products have a detectable odor. These odors, which occur at high germ concentrations, are invariably considered to be unpleasant by the consumer. The odors should therefore be referred to below as unpleasant odors. Unpleasant odors occur, for example, when washed, damp laundry is not promptly dried, for example because it has not been removed from the washing machine after completion of the washing operation.

Agents for reducing unpleasant odors are known in the prior art. Thus, for example, so-called hygienic rinses, which may be used instead of or together with a fabric softener after textiles are washed, contain germicidal cationic surfactants which reduce the number of germs and thus reduce unpleasant odors. However, the biodegradability of these cationic surfactants is questionable.

An alternative method for killing germs is the use of silver salts. Water-soluble silver salts may be used, for example, in detergents or cleaning agents, and due to their germicidal effect have an influence on the quantity of germs, and thus, on the extent of unpleasant odor of surfaces treated with the detergent or cleaning agent. However, the silver salts must be used in stabilized form in detergents or cleaning agents, so that they do not react with other components of the detergent or cleaning agent and thus become ineffective, not even during manufacture and storage of the detergent or cleaning agent. German Unexamined Patent Application DE 10 2007 025 561 A1 discloses detergents or cleaning agents having a germicidal composition based on a water-soluble silver salt and a complexing agent.

Another attempt to improve an odor impression lies not in removing unpleasant odors that are present, but, rather, to conceal them from the consumer. This is achieved by masking the unpleasant odors with odors that the consumer considers to be pleasant. A disadvantage here is that the unpleasant odors are still present, and may be noticeable again after the masking odorant perceived as pleasant has evaporated. In addition, the use of odorants in unlimited quantities is neither commercially viable nor possible under the Detergents Regulation.

Consequently, there is still a need for novel agents for reducing and/or preventing unpleasant odors. This need exists not only in the area of detergents and cleaning agents, but also in the area of cosmetics and bodily hygiene.

To meet this need, an odorant mixture is now provided which contains hexyl salicylate and at least one further salicylate, wherein the further salicylate is not 3,7,11-trimethyl-2,6,10-dodecatrien-1-yl-2-hydroxybenzoate.

Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

Odorant mixture containing hexyl salicylate and additionally at least one further salicylate, wherein the further salicylate is not 3,7,11-trimethyl-2,6,10-dodecatrien-1-yl-2-hydroxybenzoate.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

The subject matter of the present patent application relates to an odorant mixture containing hexyl salicylate and additionally at least one further salicylate, wherein the further salicylate is not 3,7,11-trimethyl-2,6,10-dodecatrien-1-yl-2-hydroxybenzoate.

Within the scope of the present patent application, an odorant mixture is understood to mean a mixture containing at least two odorant compounds. Examples of odorant compounds include synthetic products of the ester, ether, aldehyde, ketone, alcohol, and hydrocarbon types. Mixtures of various odorants are preferably used which together produce an appealing fragrance. Such fragrance oils may also contain natural odorant mixtures which are available from plant sources. Odorant mixtures according to the invention contain hexyl salicylate and at least one further salicylate, and optionally further odorant compounds.

It has surprisingly been found that an appealing odor impression as well as a reduction of unpleasant odors may be achieved by using odorant mixtures which contain hexyl salicylate and at least one further salicylate which is not 3,7,11-trimethyl-2,6,10-dodecatrien-1-yl-2-hydroxybenzoate. Hexyl salicylate and the at least one further salicylate may also be easily incorporated into existing fragrances.

The at least one further salicylate is preferably selected from the group comprising hexyl salicylate, cyclohexyl salicylate, hexenyl salicylate, methyl salicylate, benzyl salicylate, n-amyl salicylate, isoamyl salicylate, ethylhexyl salicylate, cyclooctyl salicylate, 2,2,4-(2,4,4-)trimethylcyclopentyl salicylate as an isomeric mixture, 4-isopropylcyclohexyl salicylate, cyclohexylmethyl salicylate, cycloheptyl salicylate, cyclooctyl salicylate, and cyclooct-4-enyl salicylate.

Isoamyl salicylate, cyclohexyl salicylate, benzyl salicylate, or methyl salicylate is particularly preferably used as the further salicylate. Very good results with regard to reduction of unpleasant odors have been obtained when these compounds are used as the further salicylate.

Mixtures according to the invention which contain Salicynile, CAS No. 130786-09-3, and/or cis-3-hexenyl salicylate, CAS No. 65405-77-8, are also preferred.

In addition to the hexyl salicylate and the one further salicylate, the odorant mixture preferably contains a third salicylate. This third salicylate may be selected, for example, from the above-mentioned salicylates, or may also be 3,7,11-trimethyl-2,6,10-dodecatrien-1-yl-2-hydroxybenzoate. 3,7,11-Trimethyl-2,6,10-dodecatrien-1-yl-2-hydroxybenzoate (farnesyl salicylate) is an ester of salicylic acid (2-hydroxybenzoic acid) and farnesol (3,7,11-trimethyl-2,6,10-dodecatrien-1-ol), and may be prepared from these components by means of an esterification reaction.

Accordingly, it is particularly preferred when, in addition to the hexyl salicylate and the at least one further salicylate, the odorant mixture also contains 3,7,11-trimethyl-2,6,10-dodecatrien-1-yl-2-hydroxybenzoate. The best results in reducing unpleasant odor while at the same time providing fragrance have been observed with this combination.

The use of hexyl salicylate and three, four, five, or six further salicylates is also possible.

The quantity of hexyl salicylate contained also has a significant influence on the asserted efficiency of the odorant mixtures. The odorant mixture therefore preferably contains at least 2% by weight of hexyl salicylate and preferably at least 0.5% by weight, in particular at least 1% by weight, of further salicylates (total of all further salicylates), in each case based on the overall odorant mixture.

In one preferred embodiment, the odorant mixture contains 5 to 100% by weight salicylates, in particular 10 to 30% by weight salicylates, based on the odorant mixture.

The fragrance adhesion of the remaining fragrance ingredients is preferably improved by the mixture of the salicylates.

The subject matter of the present patent application further relates to agents which include at least one surfactant from the group of nonionic or anionic surfactants, and an odorant mixture containing hexyl salicylate and additionally at least one further salicylate, wherein the further salicylate is not 3,7,11-trimethyl-2,6,10-dodecatrien-1-yl-2-hydroxybenzoate. It has been found that odorant mixtures according to the invention have the described efficiency in particular in combination with anionic and/or nonionic surfactants. Accordingly, agents according to the invention contain at least one surfactant selected from the group of anionic and nonionic surfactants.

It has surprisingly been found that agents containing hexyl salicylate and additionally at least one further salicylate, wherein the further salicylate is not 3,7,11-trimethyl-2,6,10-dodecatrien-1-yl-2-hydroxybenzoate, have the unpleasant odor-reducing and fragrancing effect not only directly after the agent is used, but also over an extended period. Without being bound to a theory, one possible explanation of this observation is that partial, possibly continuous, splitting of the salicylates occurs in the agents, and the resulting cleavage products which are released over an extended period have properties that reduce unpleasant odor, and/or have a pleasant fragrance.

The agent according to the invention is preferably a solid detergent or cleaning agent, a liquid detergent or cleaning agent, a hygienic rinse, an aftershave product, a soap product, a shower gel, a shower cream, a bath salt, a bath foam, a hair care product, a shampoo, a bodily care product, a deodorant, a cosmetic preparation, a dryer sheet, ironing water, or a wipe cloth. All of the mentioned agents, in addition to the cleaning or care of the particular surface, also allow unpleasant odors to be prevented or reduced, and thus convey a pleasant odor of the treated surface to the consumer.

As described above, the problem of formation or intensification of unpleasant odors arises in particular in conjunction with moisture, for example for textiles that are washed or damp due to other reasons, or damp, hard surfaces. Therefore, the agent according to the invention is particularly preferably a liquid detergent or cleaning agent, a solid detergent or cleaning agent, a fabric softener, a hygienic rinse, or a dryer sheet.

Depending on the type of agent according to the invention, the agent contains 0.01 to 40% by weight, preferably 0.1 to 10% by weight, of the odorant mixture according to the invention.

It has also been shown that the unpleasant odor may be reduced most effectively, and the fragrance is most prominent in the odor perception, when the agent according to the invention has a pH in the range of 5 to 9. The pH of agents according to the invention is therefore preferably in the range of 2.5 to 10.5, and in particular in the range of 5 to 9.

Preferably more than 5% by weight surfactant, preferably more than 10% by weight surfactant, and in particular more than 15% by weight surfactant, based on the agent, is contained in the agents according to the invention. Embodiments having a particularly high surfactant content may contain more than 20% by weight surfactant, more than 25% by weight surfactant, more than 30% by weight surfactant, or even more than 35% by weight surfactant. However, it is preferred that the surfactant content of the agent is below 70% by weight, preferably below 60% by weight, and in particular below 50% by weight.

Sulfonates and/or sulfates may preferably be used as anionic surfactant.

Preferably considered for surfactants of the sulfonate type are C₉₋₁₃ alkylbenzene sulfonates, olefin sulfonates, i.e., mixtures of alkene and hydroxyalkane sulfonates, as well as disulfonates, as obtained, for example, from C₁₂₋₁₈ monoolefins having a terminal or internal double bond by sulfonation with gaseous sulfur trioxide and subsequent alkaline or acidic hydrolysis of the sulfonation products. Also suitable are C₁₂₋₁₈ alkane sulfonates and the esters of α-sulfo fatty acids (ester sulfonates), for example the α-sulfonated methyl esters of hydrogenated coconut fatty acid, palm kernel fatty acid, or tallow fatty acid.

Preferred as alk(en)yl sulfates are the alkali, and in particular the sodium, salts of the sulfuric acid semi-esters of C₁₂-C₁₈ fatty alcohols, for example of coconut fatty alcohol, tallow fatty alcohol, lauryl, myristyl, cetyl, or stearyl alcohol, or the C_{10 -}C₂₀ oxo alcohols and the semi-esters of secondary alcohols having these chain lengths. The C₁₂-C₁₆ alkyl sulfates and C₁₂-C₁₅ alkyl sulfates as well as C₁₄-C₁₅ alkyl sulfates are preferred from a technical washing standpoint. In addition, 2,3-alkyl sulfates are suitable anionic surfactants.

Also suitable are fatty alcohol ether sulfates, such as the sulfuric acid monoesters of straight-chain or branched C₇₋₂₁ alcohols which are ethoxylated with 1 to 6 moles of ethylene oxide, such as 2-methyl-branched C₉₋₁₁ alcohols having an average of 3.5 moles ethylene oxide (EO), or C₁₂₋₁₈ fatty alcohols having 1 to 4 EO.

Soaps are further suitable anionic surfactants. Saturated and unsaturated fatty acid soaps, such as the salts of lauric acid, myristic acid, palmitic acid, stearic acid, (hydrogenated) erucic acid, and behenic acid, and in particular soap mixtures derived from natural fatty acids, for example coconut fatty acid, palm kernel fatty acid, olive oil fatty acid, or tallow fatty acid.

The anionic surfactants, including the soaps, may be present in the form of their sodium, potassium, magnesium, or ammonium salts. Suitable ammonium salts as counterions for the anionic surfactants are the protonated forms of choline, triethylamine, monoethanolamine, triethanolamine, or methylethylamine.

Suitable nonionic surfactants include alkoxylated fatty alcohols, alkoxylated fatty acid alkyl esters, fatty acid amides, alkoxylated fatty acid amides, polyhydroxy fatty acid amides, alkylphenol polyglycol ethers, aminoxides, alkyl polyglucosides, and mixtures thereof.

Preferably used as nonionic surfactant are alkoxylated, advantageously ethoxylated, in particular primary, alcohols preferably containing 8 to 18 C atoms and an average of 4 to 12 moles ethylene oxide (EO) per mole of alcohol, in which the alcohol radical may be linear or preferably methyl-branched in the 2-position, or may contain linear and methyl-branched radicals in the mixture, which are usually present in oxo alcohol radicals. Particularly preferred, however, are alcohol ethoxylates with linear radicals from alcohols of native origin containing 12 to 18 C atoms, for example from coconut alcohol, palm alcohol, tallow fat alcohol, or oleyl alcohol, and an average of 5 to 8 EO per mole alcohol. The preferred ethoxylated alcohols include, for example, C₁₂₋₁₄ alcohols having 4 EO or 7 EO, C₉₋₁₁ alcohols having 7 EO, C₁₃₋₁₅ alcohols having 5 EO, 7 EO, or 8 EO, C₁₂-₁₈ alcohols having 5 EO or 7 EO, and mixtures thereof. The stated ethoxylation numbers represent statistical average values, which for a particular product may be a whole number or a fractional number. Preferred alcohol ethoxylates have a narrow homolog distribution (narrow range ethoxylates (NRE)). In addition to these nonionic surfactants, fatty alcohols having greater than 12 EO may also be used. Examples of such are tallow fat alcohol having 14 EO, 25 EO, 30 EO, or 40 EO. Nonionic surfactants which contain EO and PO groups together in the molecule are also usable according to the invention. Also suitable is a mixture of a (more heavily) branched ethoxylated fatty alcohol and an unbranched ethoxylated fatty alcohol, such as a mixture of a C₁₆₋₁₈ fatty alcohol with 7 EO and 2-propylheptanol with 7 EO. The agent according to the invention particularly preferably contains a C₁₂₋₁₈ fatty alcohol with 7 EO or a C₁₃₋₁₅ oxo alcohol with 7 EO or 8 EO as nonionic surfactant.

One advantage of the present invention is that the use of germicidal cationic surfactants may be reduced, or that these surfactants may even be dispensed with entirely. Accordingly, agents according to the invention preferably contain less than 5% by weight, preferably less than 1% by weight, of cationic surfactant, and in particular contain no cationic surfactant.

The subject matter of the present patent application relates to the use of an odorant mixture which includes hexyl salicylate and at least one further salicylate, wherein the further salicylate is not 3,7,11-trimethyl-2,6,10-dodecatrien-1-yl-2-hydroxybenzoate, and an agent which contains such an odorant mixture, for reducing unpleasant odors.

In this regard, the odorant mixture according to the invention is preferably contained in liquid detergents or cleaning agents, solid detergents or cleaning agents, fabric softeners, hygienic rinses, or dryer sheets.

In a further aspect, the present invention relates to a method for producing a detergent or cleaning agent, hygienic rinse, or dryer sheet which reduces unpleasant odors, in which an odorant mixture which contains hexyl salicylate and at least one further salicylate, wherein the further salicylate is not 3,7,11-trimethyl-2,6,10-dodecatrien-1-yl-2-hydroxybenzoate, is mixed with further customary ingredients of detergents or cleaning agents, fabric softeners, hygienic rinses, or dryer sheets.

If the agent according to the invention is a dryer sheet, the odorant mixture according to the invention is preferably sprayed onto the fabric of the dryer sheet. If the agent according to the invention is liquid, the odorant mixture according to the invention is preferably stirred in with other liquid components of the agent.

The invention further relates to a method for producing a solid detergent or cleaning agent which reduces unpleasant odors, in which an odorant mixture according to the invention is applied to a carrier in powdered form, and the resulting product is mixed with further ingredients of the detergent or cleaning agent.

Silica, sodium carbonate, sodium sulfate, zeolite, sodium silicate, aluminosilicate, or a mixture including a plurality of the components mentioned here is preferably used as the carrier in powdered form.

In another aspect, the invention relates to a method for producing a solid detergent or cleaning agent which reduces unpleasant odors, in which an odorant mixture according to the invention is mixed into nonionic surfactant, and the resulting product is mixed with further ingredients of the detergent or cleaning agent. The mixture produced in this method, containing the odorant mixture according to the invention and nonionic surfactant, is preferably applied in one of the post-addition steps.

The agent according to the invention may contain further ingredients in addition to the odorant mixture according to the invention and the contained surfactant.

Liquid detergents or cleaning agents may, for example, contain additional ingredients which further improve the application-related and/or esthetic properties of the detergent or cleaning agent. Within the scope of the present invention, a detergent or cleaning agent according to the invention preferably additionally contains one or more substances from the group of builders, bleaching agents, bleach catalysts, bleach activators, enzymes, electrolytes, pH adjusters, fragrances, fragrance carriers, fluorescence agents, dyes, hydrotopes, foam inhibitors, silicone oils, shrinkage inhibitors, anti-crease agents, dye transfer inhibitors, antioxidants, preservatives, corrosion inhibitors, anti-static agents, bittering agents, ironing aids, repellent and impregnation agents, skin care active substances, swelling and anti-slip agents, softening components, and UV absorbers.

### EXAMPLE

TABLE 1
Odorant mixtures

| | Hexyl salicylate | Isoamyl salicylate | Cyclohexyl salicylate |
|---|---|---|---|
| D1 | 125 | 25 | 10 |
| D2 | 150 | — | 10 |
| V1 | 160 | — | — |

[Values are expressed as weight ratios]

The odorant mixtures D1 and D2 according to the invention contain at least one further salicylate in addition to hexyl salicylate. In comparative example V1, no further salicylate is added to the hexyl salicylate.

The odorant mixtures from Table 1 were mixed into the liquid detergent below in a quantity of 1.5% by weight, based on 100% by weight agent plus the odorant mixture.

TABLE 2
Liquid detergent

| | Agent 1 | Agent 2 | Agent 3 |
|---|---|---|---|
| | containing | containing | containing |
| | D1 | D2 | V1 |
| C₁₂₋₁₈ROH (7 EO) | 12.0 | 12.0 | 12.0 |
| C₁₂₋₁₄ dimethylamine oxide | 2.0 | 2.0 | 2.0 |
| C₁₂₋₁₈ fatty acid | 2.5 | 2.5 | 2.5 |
| H₂O₂ (35%) | 0.2 | 0.2 | 0.2 |
| Citric acid · H₂O | 0.1 | 0.1 | 0.1 |
| Sodium hydroxide solution | 3 | 3 | 8.2 |
| (50%) for setting the pH to: | | | |
| D1, D2, or V1 | 1.5 | 1.5 | 1.5 |
| Water | To make | To make | To make |
| | 100 | 100 | 100 |

[All quantities are expressed in % by weight active substance, based on the overall composition]

Two samples each of a polyester fabric and a cotton fabric were treated with a dosage of 80 g of agents 1 to 3 in a Miele Novotronic W526 apparatus, at a water level of 7.6 L and a temperature of 20° C. 100 g each of an alkaline and an acidic synthetic perspiration were added in each wash test.

The test fabrics were rinsed with clean water and left for 24 hours in a closed washing machine.

The test fabrics were subsequently independently assessed for odor by three trained personnel, in each case the fragrance odor and the unpleasant odor being determined. The ratio of unpleasant odor to fragrance odor was determined. The intensities of the odors were assessed on a scale of 0 to 10 (0=not perceivable, 10=extreme odor impression).

TABLE 3
Results of fragrance odor and unpleasant odor determination

| | Agent 1 | Agent 2 | Agent 3 |
|---|---|---|---|
| | containing D1 | containing D2 | containing V1 |
| Unpleasant odor | 2 | 3 | 4 |

(Average value of 3 separate single determinations; 0 = no unpleasant odor; 6 = extremely strong unpleasant odor)

A distinct reduction in the unpleasant odor of the textiles was determined by trained test personnel for use of agents 1 and 2 according to the invention. In contrast, only a strong unpleasant odor was perceivable for use of agent 3 not according to the invention.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. An odorant mixture comprising hexyl salicylate and additionally at least one further salicylate, wherein the further salicylate is not 3,7,11-trimethyl-2,6,10-dodecatrien-1-yl-2-hydroxybenzoate.

2. A mixture according to claim 1, wherein the further salicylate in the mixture is selected from the group consisting of hexyl salicylate, cyclohexyl salicylate, hexenyl salicylate, methyl salicylate, benzyl salicylate, n-amyl salicylate, isoamyl salicylate, 2,2,4-(2,4,4-)trimethylcyclopentyl salicylate as an isomeric mixture, ethylhexyl salicylate, cyclooctyl salicylate, 4-isopropylcyclohexyl salicylate, cyclohexylmethyl salicylate, cycloheptyl salicylate, cyclooctyl salicylate, and cyclooct-4-enyl salicylate.

3. A mixture according to claim 1, wherein in addition to the hexyl salicylate and the at least one further salicylate, the mixture contains a third salicylate.

4. A mixture according to claim 1, wherein the mixture contains at least 2% by weight hexyl salicylate, based on the mixture.

5. A mixture according to claim 1, wherein the fragrance adhesion of the remaining fragrance ingredients is improved by the mixture of the salicylates.

6. A mixture according to claim 1, wherein the mixture includes 5 to 100% by weight salicylates, based on the mixture.

7. An agent comprising at least one surfactant selected from the group of nonionic or anionic surfactants and a mixture according to claim 1.

8. An agent according to claim 7, wherein the agent is a solid detergent or cleaning agent, a liquid detergent or cleaning agent, a hygienic rinse, an aftershave product, a soap product, a shower gel, a shower cream, a bath salt, a bath foam, a hair care product, a shampoo, a bodily care product, a deodorant, a cosmetic preparation, a dryer sheet, ironing water, or a wipe cloth.

9. An agent according to claim 7, wherein the agent contains 0.01 to 40% by weight, of the mixture.

10. An agent according to claim 7, wherein the agent has a pH in the range of 2.5 to 10.5.

11. An agent according to claim 7, wherein the agent contains more than 5% by weight of surfactant.

12. An agent according to claim 7, wherein the agent contains less than 5% by weight, of cationic surfactant.

13. A method for producing a detergent or cleaning agent, fabric softener, hygienic rinse, or dryer sheet which reduces unpleasant odors, in which a mixture according to claim 1 is mixed with further customary ingredients of detergents or cleaning agents, fabric softeners, hygienic rinses, or dryer sheets.
